(19) United States
(12) Patent Application Publication
(10) Pub. No.: US 2025/0339183 A1
(43) Pub. Date: Nov. 6, 2025

(54) BONE SECURING SYSTEMS AND METHODS

(71) Applicant: GetSet Surgical SA, Epalinges (CH)

(72) Inventor: Didier Guillon-Cottard, Brignais (FR)

(73) Assignee: GetSet Surgical SA, Epalinges (CH)

(21) Appl. No.: 19/105,769

(22) PCT Filed: Jul. 17, 2023
(86) PCT No.: PCT/IB2023/057288

§ 371 Date: Feb. 21, 2025

Related U.S. Application Data
(60) Provisional application No. 63/374,259, filed on Sep. 1, 2022.

(51) Int. Cl. A61B 17/70

(52) U.S. Cl. CPC A61B 17/7034; A61B 17/7038

(57) ABSTRACT

A bone shank can include a shank head and an extension extending from the shank head. The shank head can include an engaging surface that faces away from the extension. A head assembly can be designed to secure the shank head to a rod. The head assembly can include an engaging surface that is shaped and positioned to engage the engaging surface of the shank head to limit pivotal movement of the shank relative to the engaging surface of the head assembly, but to allow some pivotal movement of the shank relative to the engaging surface of the head assembly. The engaging surface of the head assembly can be an engaging surface of a head clamp in an assembly head of the head assembly. The engaging surface of the head clamp may face distally toward the shank head while the clamp is positioned in the assembly head.

### FIELD

This disclosure relates to bone securing systems and methods.

### BACKGROUND

Spinal fixation procedures utilizing pedicle screws and rod-based fixation assemblies can be used to correct spinal conditions such as degenerative disc disease, spondylolisthesis, spinal deformities, or other spinal conditions through minimally invasive or invasive spinal surgery. For example, two or more bone anchor assemblies may be secured into bone structures of a patient's vertebrae with connecting rods secured between adjacent bone anchor assemblies in order to stabilize one or more vertebral joints of a patient. These connecting rods typically run longitudinally along the length of the patient's spine between adjacent bone anchor assemblies. However, connecting rods can be arranged in a variety of positions and/or configurations (including the use of multiple connecting rods and/or crossbars, where desired) in view of a patient's specific anatomy and/or a specific spinal correction.

### SUMMARY

According to one example, a bone securing system can include a shank that is designed to be secured to bone. The shank can include a shank head and an extension extending from the shank head. Also, the shank head can include an engaging surface. The bone securing system can also include a head assembly, which can include an assembly head and a head clamp. The assembly head can define a central hole therethrough extending in a proximal direction and in a distal direction that is opposite the proximal direction. The assembly head can define a channel transverse to the central hole. Also, the assembly head can include tabs extending in the proximal direction on opposite sides of the channel and the central hole. The head clamp can be designed to be positioned at least partially within the central hole. The head clamp can be designed to receive the shank head and to clamp onto the shank head to secure the shank head in position at least partially in the assembly head with the extension extending away from the head assembly. The head clamp can include an engaging surface that is shaped and positioned to engage the engaging surface of the shank head to limit pivotal movement of the shank relative to the head clamp to a movement pattern. At least a portion of the engaging surface of the head clamp can face in the distal direction toward the shank head.

According to another example, a shank can be designed to be secured to bone. The shank can include a shank head and an extension extending from the shank head. The shank head can include an engaging surface that faces away from the extension. The bone securing system can also include a head assembly designed to secure the shank head to a rod. The head assembly can include an engaging surface that is shaped and positioned to engage the engaging surface of the shank head to limit pivotal movement of the shank relative to the engaging surface of the head assembly, but to allow some pivotal movement of the shank relative to the engaging surface of the head assembly. The engaging surface of the head assembly can face toward the engaging surface of the shank head.

According to yet another example, a bone securing system can include a shank designed be secured to bone. The shank can include a shank head and an extension extending from the shank head. The shank head can include an engaging surface that faces away from the extension. The bone securing system can also include multiple different head assemblies that each allow a different pivotal movement pattern between the head assembly and the shank. A first head assembly of the head assemblies can include a first engaging surface that is shaped and positioned to engage the engaging surface of the shank head to limit pivotal movement of the shank relative to the first engaging surface of the first head assembly to a first movement pattern. The first engaging surface of the first head assembly can face toward the engaging surface of the shank head.

According to yet another example, a technique can include selecting a selected head clamp from multiple different available head clamps. The different available head clamps can be designed to produce different pivotal movement patterns between head assemblies (in which the head clamps are secured) and shanks (which are secured to the head assemblies using the head clamps). The selected head clamp can be designed to produce a selected pivotal movement pattern. The technique can also include securing a head assembly on a shank head of a shank in a motion limiting configuration. The shank can be designed to be secured in bone. The head assembly can include the selected head clamp positioned at least partially in an assembly head in the motion limiting configuration. The head assembly can be configured to secure the shank to a rod. The securing of the head assembly can include receiving at least a portion of the shank head in the selected head clamp. Also, the selected head clamp can include an engaging surface that is shaped and positioned to engage the engaging surface of the shank head to limit pivotal movement of the shank relative to the selected head clamp to the selected pivotal movement pattern while in the motion limiting configuration. The technique can further include pivoting the head assembly and the shank relative to each other within the selected pivotal movement pattern while the selected head clamp and the shank are in the motion limiting configuration. The pivoting can include pivoting at least a portion of the engaging surface of the shank head away from at least a portion of the engaging surface of the selected head clamp while the at least a portion of the engaging surface of the shank head and the at least a portion of the engaging surface of the head clamp face toward each other.

This Summary is provided to introduce a selection of concepts in a simplified form. The concepts are further described below in the Detailed Description. This Summary is not intended to identify key features or essential features of the claimed subject matter, nor is it intended to be used to limit the scope of the claimed subject matter. Similarly, the invention is not limited to implementations that address the particular techniques, tools, environments, disadvantages, or advantages discussed in the Background, the Detailed Description, or the attached drawings.

### BRIEF DESCRIPTION OF THE DRAWINGS

FIG. 1 is a top perspective view of a bone securing system (with toward the top being in a proximal direction and toward the bottom being in a distal direction, as used herein, though terms such as top and bottom should not be considered to be absolute directions).

FIG. 2 is a top perspective view of a bone anchor assembly from the bone anchor system of FIG. 1 secured to bone.

FIG. 3 is a top perspective exploded view of the bone anchor assembly of FIG. 2.

FIG. 4 is a right-side perspective view of the bone anchor assembly of FIG. 2 in a locked configuration. A left side view would be a mirror image of the right-side view.

FIG. 5 is a cutaway sectional view taken along line 5-5 of FIG. 4.

FIG. 6 is a right-side view of the bone anchor assembly of FIG. 2 in a separated open configuration. A left side view would be a mirror image of the right-side view.

FIG. 7 is a cutaway sectional view taken along line 7-7 of FIG. 6.

FIG. 8 is a right-side view of the bone anchor assembly of FIG. 2 in a mounted open configuration. A left side view would be a mirror image of the right-side view.

FIG. 9 is a cutaway sectional view taken along line 9-9 of FIG. 8.

FIG. 10 is a right-side view of the bone anchor assembly of FIG. 2 in a mounted closed configuration. A left side view would be a mirror image of the right-side view.

FIG. 11 is a cutaway sectional view taken along line 11-11 of FIG. 10.

FIG. 12 is a bottom perspective view of an assembly head of the bone anchor assembly of FIG. 2.

FIG. 13 is a top perspective view of a transverse uniaxial head clamp of the bone anchor assembly of FIG. 2.

FIG. 14 is a bottom perspective view of the head clamp of FIG. 13.

FIG. 15 is another top perspective view of the head clamp of FIG. 13.

FIG. 16 is a front view of the head clamp of FIG. 13. A rear view would be a mirror image of the front view.

FIG. 17 is a right-side view of the head clamp of FIG. 13. A left-side view would be a mirror image of the right-side view.

FIG. 18 is a top view of the head clamp of FIG. 13.

FIG. 19 is a bottom view of the head clamp of FIG. 13.

FIG. 20 is a top perspective view of an inline uniaxial head clamp that can be used in the bone anchor assembly in place of the transverse uniaxial head clamp of FIG. 13.

FIG. 21 is a bottom perspective view of the head clamp of FIG. 20.

FIG. 22 is a top view of the head clamp of FIG. 20.

FIG. 23 is a bottom view of the head clamp of FIG. 20.

FIG. 24 is a top perspective view of a monoaxial head clamp that can be used in the bone anchor assembly in place of the transverse uniaxial head clamp of FIG. 13.

FIG. 25 is a bottom perspective view of the head clamp of FIG. 24.

FIG. 26 is a top view of the head clamp of FIG. 24.

FIG. 27 is a bottom view of the head clamp of FIG. 24.

FIG. 28 is a top perspective view of a polyaxial head clamp that can be used in the bone anchor assembly in place of the transverse uniaxial head clamp of FIG. 13.

FIG. 29 is a bottom perspective view of the head clamp of FIG. 28.

FIG. 30 is a top view of the head clamp of FIG. 28.

FIG. 31 is a bottom view of the head clamp of FIG. 28.

FIG. 32 is a left-side view of the bone anchor assembly of FIG. 2, with the shank pivoted relative the head assembly, instead of aligned with the head assembly as in the previous figures.

FIG. 33 is a cutaway sectional view taken along line 33-33 of FIG. 32.

FIG. 34 is a front view of another bone anchor assembly similar to the bone anchor assembly of FIG. 2 in a mounted closed configuration with the shank and the head assembly aligned, but including the inline uniaxial head clamp of FIGS. 20-23 instead of the transverse uniaxial head clamp of FIGS. 13-19.

FIG. 35 is a cutaway sectional view taken along line 35-35 of FIG. 34.

FIG. 36 is a front view of the bone anchor assembly of FIG. 34 in the mounted closed configuration with the shank and the head assembly pivoted relative to each other.

FIG. 37 is a cutaway sectional view taken along line 37-37 of FIG. 36.

FIG. 38 is a view like FIG. 34, but with an alternative inline uniaxial head clamp.

FIG. 39 is a cutaway sectional view taken along line 39-39 of FIG. 38.

FIG. 40 is a flowchart illustrating an apparatus assembly technique.

The description and drawings may refer to the same or similar features in different drawings with the same reference numbers.

### DETAILED DESCRIPTION

Example implementations of the present disclosure may be understood by

reference to the drawings. The components of the present disclosure, as generally described and illustrated in the Figures herein, could be arranged and designed in a wide variety of different configurations. Thus, the following more detailed description of the examples of the apparatus and method, as represented in the Figures, is not intended to limit the scope of the present disclosure, as claimed in this or any other application claiming priority to this application, but is merely representative of example implementations of the present disclosure.

Standard medical directions, planes of reference, and descriptive terminology are employed in this specification. For example, anterior means toward the front of the body. Posterior means toward the back of the body. Superior means toward the head. Inferior means toward the feet. Medial means toward the midline of the body. Lateral means away from the midline of the body. Ipsilateral means on the same side of the body. Contralateral means on the opposite side of the body. A sagittal plane divides a body into right and left portions. A midsagittal plane divides the body into bilaterally symmetric right and left halves. A coronal plane divides a body into anterior and posterior portions. A transverse plane divides a body into superior and inferior portions. These descriptive terms may be applied to an animate or inanimate body. As used herein, the term “proximal,” “proximal direction,” “proximally” and similar terms generally refer to directions going along the components away from the leading tip of the shank (the end of the shank farthest from the shank head). As used herein, the terms “distal,” “distal direction,” “distally” and similar terms generally refer to directions going along the components toward the leading tip of the shank. For example, with regard to the components of the head assembly in the illustrated examples, the distal direction extends generally from the set screw toward the shank head, such as in a direction that is parallel to an axis about which a set screw or other rotating securing device is turned to secure a rod in the head assembly, and the distal direction is opposite the proximal direction. As another example, for a threaded shank, the proximal direction extends along the longitudinal axis of the shank (around which the shank can turn to screw into or out of bone) from the tip of the shank toward the head of the shank, and the distal direction is opposite to the proximal direction. Thus, a proximal direction for the shank may be different from the proximal direction of the assembly head when the shank is pivoted relative to the assembly head.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

### I. Basic Bone Securing System Structure

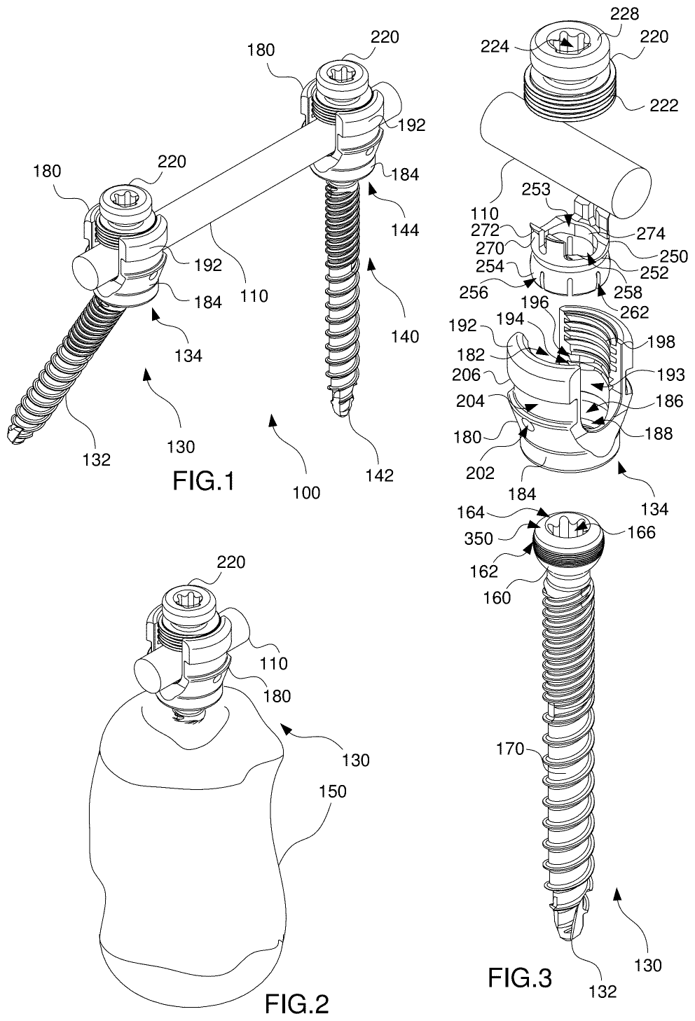

Referring to FIG. 1, bone securing system 100 can secure different portions of a rod 110 to multiple different bone regions (which may be regions of different bones or different regions of the same bone), which can inhibit movement of the different bone regions relative to each other. As an example, the different bone regions may be pedicles of different vertebrae of a spine. For example, the bone securing system 100 can include first and second bone anchor assemblies that can include different head assemblies to allow for different pivotal movement patterns of shanks in those bone anchor assemblies relative to the assembly heads of those bone anchor assemblies.

As an example, a movement pattern may be a monoaxial movement pattern, wherein an assembly is designed to inhibit substantial pivoting of a shank around axes other than a longitudinal axis of the shank, such as a bone screw that can rotate around its longitudinal axis to be screwed into or out of bone, but is inhibited from substantial pivoting around other axes. As another example, a movement pattern may be a polyaxial movement pattern, wherein an assembly is designed to allow substantial pivoting of a shank around multiple axes other than a longitudinal axis of the shank, though possibly limiting the extent of such movement, such as allowing rotation of the shank around its longitudinal axis and allowing pivotal movement of plus or minus thirty degrees around any axis that is perpendicular to the longitudinal axis of the shank, but inhibiting pivotal movement beyond those limits. As another example, a movement pattern may be a uniaxial movement pattern, wherein an assembly is designed to allow substantial pivoting of a shank around a single axis other than a longitudinal axis of the shank, though possibly limiting the extent of such pivoting movement, such as allowing rotation of the shank around its longitudinal axis and allowing pivotal movement of thirty degrees around a single axis that is perpendicular to the longitudinal axis of the shank, but inhibiting pivotal movement beyond those limits. For example, for a transverse uniaxial pivoting movement pattern, this single axis may be parallel to a longitudinal axis of the rod, so that the assembly is designed to allow the shank to pivot in a transverse direction substantially in a plane that is perpendicular to the longitudinal axis of the rod. As another example, for an inline uniaxial pivoting movement pattern, this single axis may be perpendicular to the longitudinal axis of the rod, so that the assembly is designed to allow the shank to pivot in an inline direction substantially in a plane that is parallel to the longitudinal axis of the rod. Such a plane may or may not intersect the longitudinal axis of the rod.

For example, the bone securing system 100 of FIG. 1 can include a first bone anchor assembly 130, which can include a shank 132 and a head assembly 134, which can limit pivotal movement of the shank 132 relative to the head assembly 134 to a first movement pattern, which can be a transverse uniaxial movement pattern, as illustrated by the shank 132 in FIG. 1 having been pivoted in a plane that is perpendicular to the longitudinal axis of the rod 110. The bone securing system 100 of FIG. 1 can also include a second bone anchor assembly 140, which can include a shank 142 and a head assembly 134, which can limit pivotal movement of the shank 142 relative to the head assembly 144 to a second movement pattern that is different from the first movement pattern. For example, the second movement pattern may be different from the first movement pattern (such as an inline uniaxial movement pattern, a monoaxial movement pattern, or a polyaxial movement pattern). Different head assemblies allowing different shank movement patterns may be chosen by medical practitioners during surgical procedures, such as depending on the needs arising from different patients and different surgical procedures.

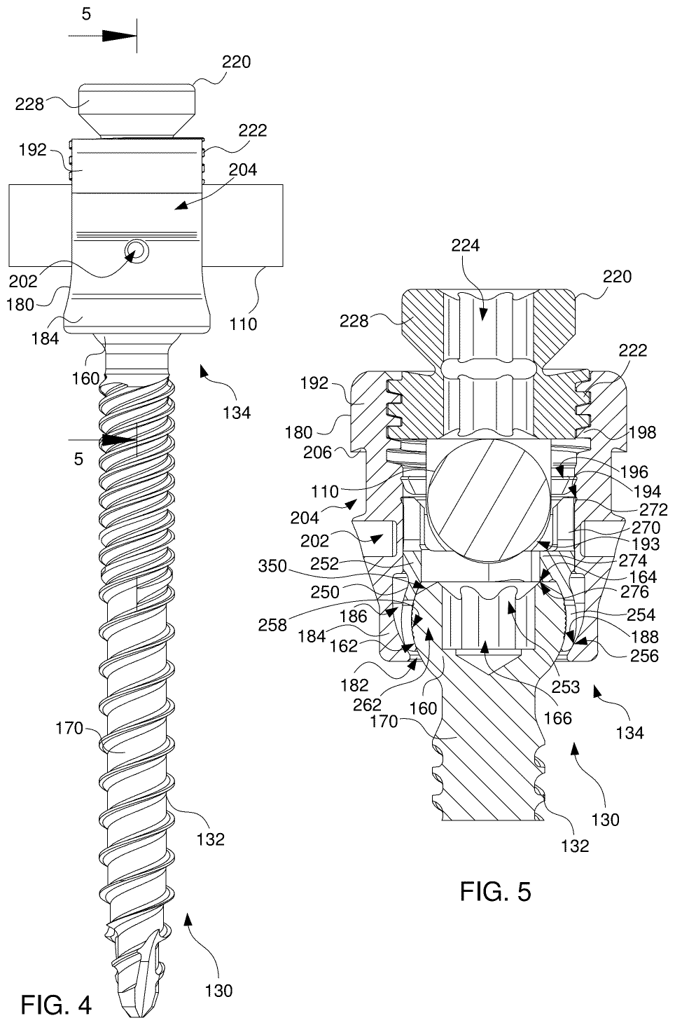

Referring now to FIGS. 1-5, the first bone anchor assembly 130 of FIG. 1 will be discussed in more detail. The other bone anchor assemblies can have the same features as the first bone anchor assembly 130, except that the clamps in those bone anchor assemblies may be configured to produce different types of pivotal movement patterns, as discussed herein.

As illustrated in FIG. 2, the shank 132 of the first bone anchor assembly 130 can be anchored in or otherwise secured to bone 150. The illustration of bone 150 is not intended to be the shape of any particular type of bone, but is included for illustration purposes. As noted above, the bone securing system 100 may be used with different types of bones, such as with vertebrae. Also, as illustrated in FIGS. 2-5, the shank 132 may be aligned with the head assembly 134, rather than pivoted at an angle to the head assembly 134, as illustrated with the first bone anchor assembly 130 FIG. 1.

The shank 132 can include a shank head 160, which can include a rounded clamped surface 162, which may form a partial sphere. A proximal end of the shank head 160 can form an engaging surface 164, which can be a generally annular shape. The engaging surface 164 can face proximally. For example, the engaging surface 164 may be an inner edge of a surface that slopes distally as it extends outwardly from the inner edge. Alternatively, the engaging surface 164 may be some other shape, such as a planar surface that extends in a plane that is perpendicular to a rotational and longitudinal axis of the shank 132. The shank head 160 can also define a tool-receiving aperture 166 that extends distally into the shank head 160 from its proximal end, with the engaging surface 164 extending around the tool-receiving aperture 166. The shank 132 can also include an extension 170, which can extend distally away from a distal end of the shank head 160. Thus, the engaging surface 164 can face away from the extension 170. For example, if the shank 132 is a bone screw, the extension 170 can be threaded, and different portions of the extension may have different thread features, as illustrated. Thus, the shank 132 can be designed to be screwed into and out of bone as it is rotated around its longitudinal axis. However, the extension of the shank may have different features, depending on how the shank is designed to be secured to a bone.

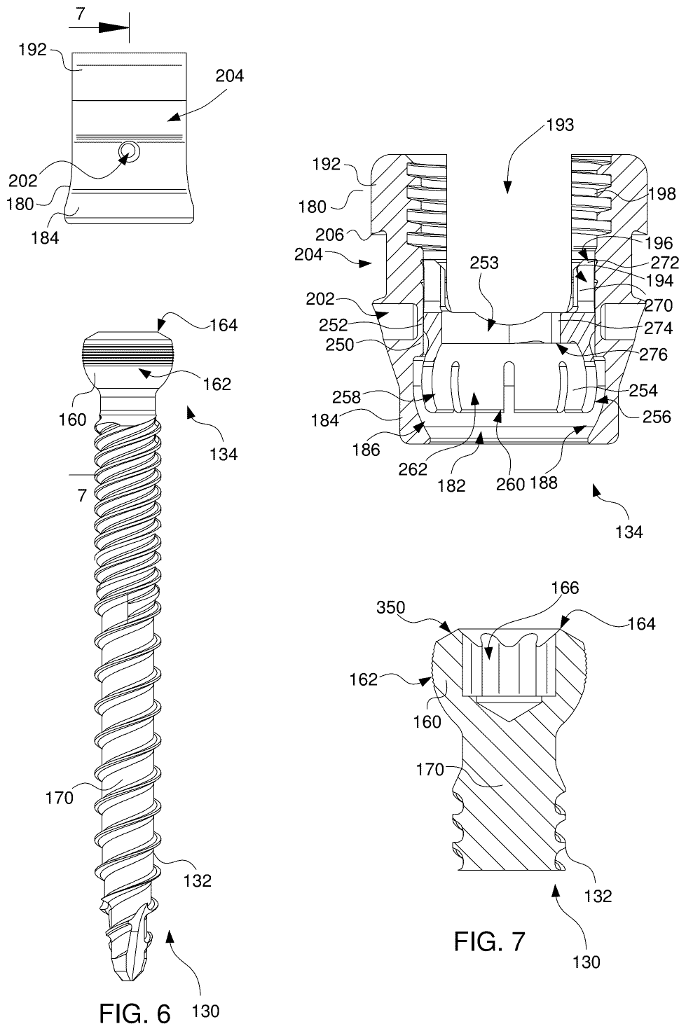

Referring still to FIGS. 1-5 and also to FIGS. 6-12, the head assembly 144 can include an assembly head 180. The assembly head 180 can define a central hole 182 extending in an axial direction that can be a proximal and distal direction through the assembly head 180. The assembly head 180 can include a body 184 that defines a distal cavity 186 that is part of the central hole 182. The body 184 can extend fully around the distal cavity 186 of the central hole 182. An inner surface of the body 184 can slope inwardly as it extends towards a distal end of the body 184, so that an inner diameter of the body 184 can decrease toward the distal end of the body 184. This sloping inner surface of the body 184 can be a clamp engaging surface 188, as is discussed more below. The inner surface of the body 184 can extend inwardly above an enlarged area of the cavity to form a shoulder.

The assembly head 180 can also include a pair of tabs 192, which can be curved around a central axis of the assembly head 180 and can extend proximally from the body 184 on opposite sides of a channel 193, which can extend transversely to the central hole 182 and intersect the central hole 182. The channel 193 can extend distally from the proximal end of the assembly head 180, and can be rounded at its distal base. The channel 193 can be sized to receive the rod 110 therein. Inner surfaces of the tabs 192 can face each other. These inner surfaces of the tabs 192 can define a distal aperture or distal groove 194 and a proximal aperture or proximal groove 196, both extending in a circumferential direction around the inner surfaces of the tabs 192. The inner surfaces of the tabs 192 can also define female threads 198 positioned proximally from the proximal groove 196. Outer surfaces of the tabs 192 can also define one or more apertures. For example, a hole 202 and a ring-shaped, circumferentially extending recess 204 can extend into the outer surface of each tab 192. A proximal shoulder 206 defining a proximal edge of each recess 204 can be undercut, so that the shoulder extends distally as it extends out from a central portion of the assembly head 180.

The head assembly 144 can also include a set screw 220, which can include a distal portion having male threads 222 that are designed to mate with the female threads 198 of the assembly head 180. The set screw 220 can define a central tool-receiving aperture 224 extending in a proximal-distal direction through the set screw 220. A proximal portion of the set screw 220 can be a break-off head 228, which can be designed to break off from the distal portion of the set screw when a predetermined amount of torque is applied to the break-off head 228 during tightening of the set screw 220.

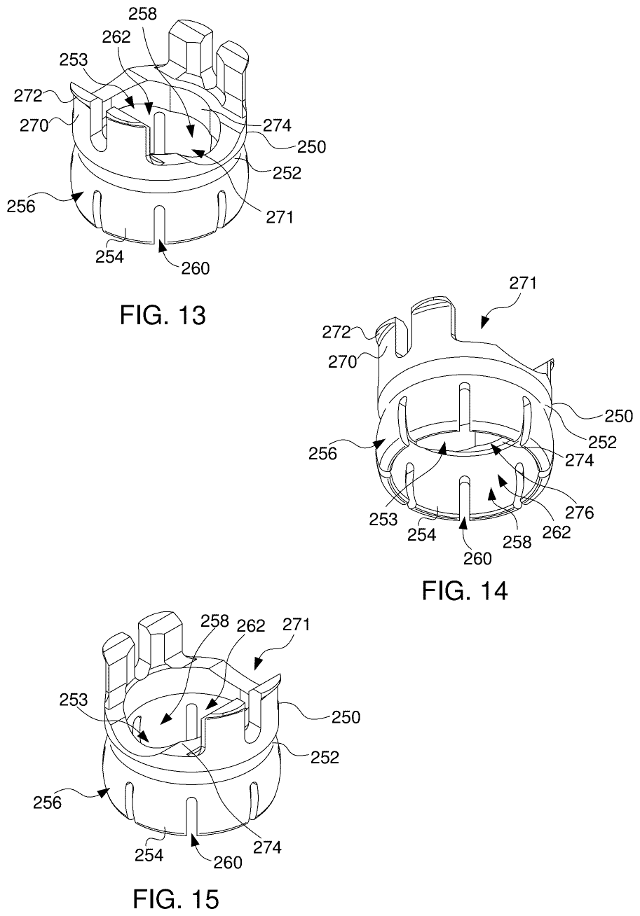

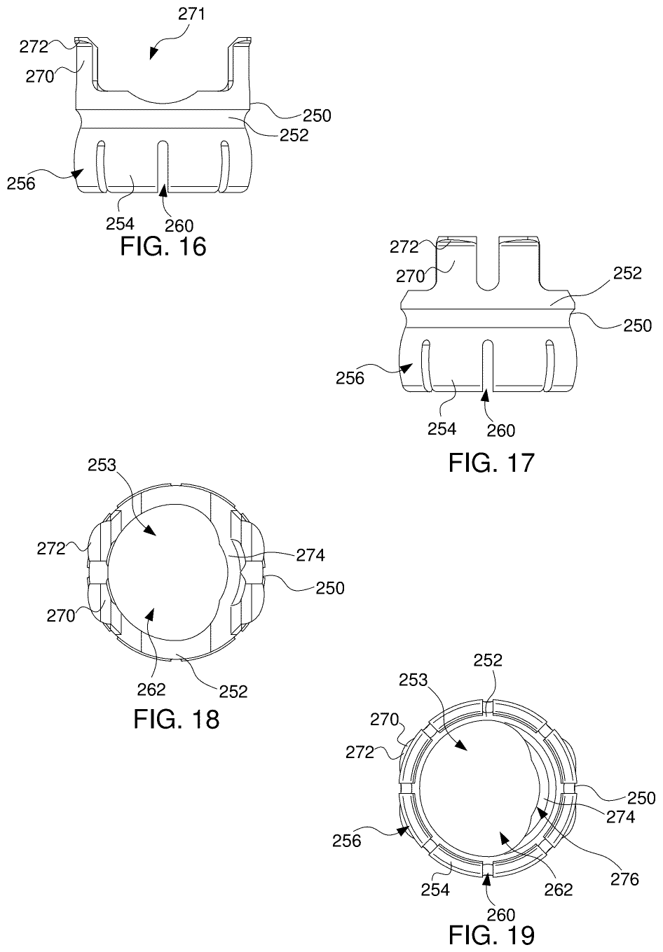

Referring still to FIGS. 12 and also to FIGS. 13-19, the head assembly 144 can also include a head clamp 250. The clamp 250 can be a collar, which can include a main body 252, which can be circular and can define a central axial hole 253 therein. The clamp 250 can include fingers 254 extending distally from the main body 252, with the fingers 254 being separated by gaps 260 that extend proximally into the clamp 250 between the fingers 254. The fingers 254 can be circumferentially spaced around the main body 252, and each finger 254 can extend radially out and then radially back in as it extends distally away from the main body 252, so that each finger 254 can include a convex outer engaging surface 256 and a concave inner clamping surface 258, with the concave inner clamping surfaces 258 of the fingers 254 and an inner portion of the main body 252 defining a socket 262 that is designed to receive the shank head 160 with the fingers 254 wrapping around the shank head 160. The socket 262 may form a partial spherical shape. The convex outer engaging surfaces 256 of the fingers 254 can be designed to engage the clamp engaging surface 188 of the assembly head 180 in some positions, as discussed below.

The clamp 250 can also include flanges 270 extending proximally from the main body 252. A pair of flanges 270 can extend adjacent to each of the tabs 192 of the assembly head 180, defining a channel 271 between a first pair of the flanges 270 and a second pair of the flanges 270. The channel 271 can align with the channel 193 between the tabs 192 of the assembly head 180. Also, a protrusion 272 can extend radially out from a proximal end of each flange 270. The protrusions 272 can extend into the proximal groove 196 or the distal groove 194 of the assembly head 180, depending on whether the assembly head 180 and the clamp 250 are in an open or closed configuration relative to each other, as discussed below.

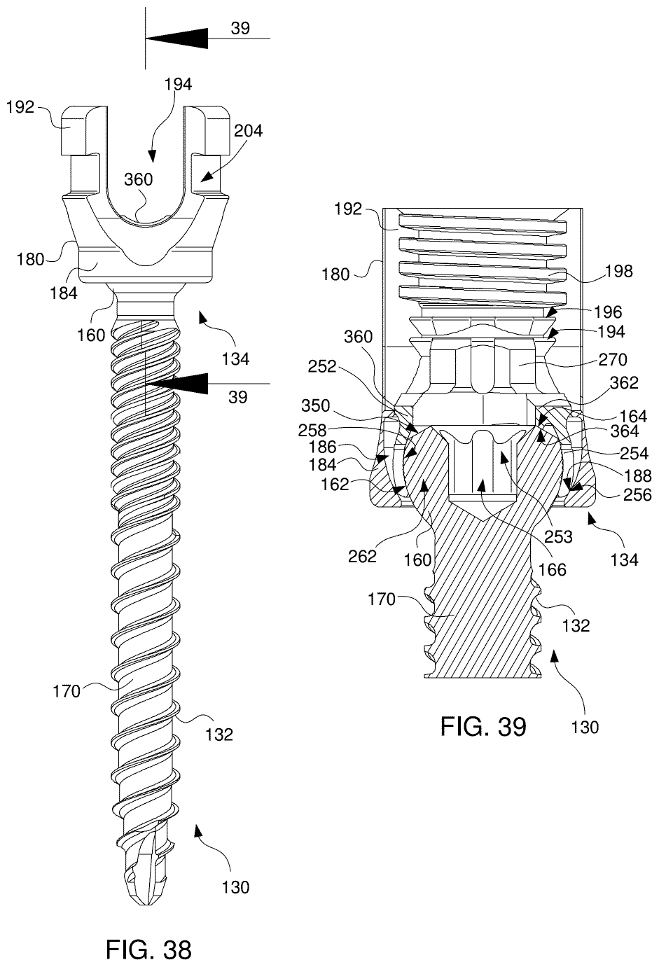

Different clamps can have different engaging surfaces to engage with (or not engage with) the engaging surface 164 of the shank head 160. In the example, the clamp 250 is a transverse uniaxial clamp, which limits pivotal movement of the shank 142 to a transverse uniaxial movement pattern. As such, the clamp 250 can include a motion limiting protrusion 274 that protrudes radially inward from the main body 252 of the clamp 250. The motion limiting protrusion 274 can include a distally facing shank engaging surface 276, which can be on a distally facing shoulder formed by the motion limiting protrusion 274. For example, the shank engaging surface 276 may be an inner edge of a distally facing planar surface, or it may be some other shape, such as an entire planar surface or non-planar surface or a portion of a planar surface or non-planar surface, depending on the configuration of the clamp 250 and the shank 142 (for example, see the discussion below regarding FIGS. 38-39). Thus, the engaging surfaces of the clamp 250 and the shank 142 may engage each other in a point contact, a line contact, or a broader surface contact. The motion limiting protrusion 274 can form a partial ring that extends inwardly from a portion of the main body 252 adjacent to one pair of the flanges 270, which can also be adjacent to one of the tabs 192 of the assembly head 180. The motion limiting protrusion 274 can be shaped so that its shank engaging surface 276 can engage the engaging surface 164 of the shank head 160 to limit pivotal movement of the shank 142 relative to the clamp 250 and the assembly head 180 to a transverse uniaxial movement pattern, and to inhibit movements outside of such a pattern. As discussed below, this movement pattern may not be produced by the clamp 250 in all positions. For example, this limited movement pattern may be produced in a mounted closed position, but it may not be produced in a separated configuration, a mounted open configuration, or a mounted locked configuration. Such configurations are discussed below in a discussion of use of the bone securing system.

### II. Alternative Clamps for Different Pivotal Movement Patterns

Different clamps can be used with the other components of the bone securing system 100 to produce different movement patterns. Such different movement patterns can be produced my only changing the clamps, without making changes to the other components of the bone securing system 100. Some examples of such different clamps are discussed below. As discussed above, the transverse uniaxial clamp 250, illustrated in FIGS. 13-19, can limit movement to transverse uniaxial movement.

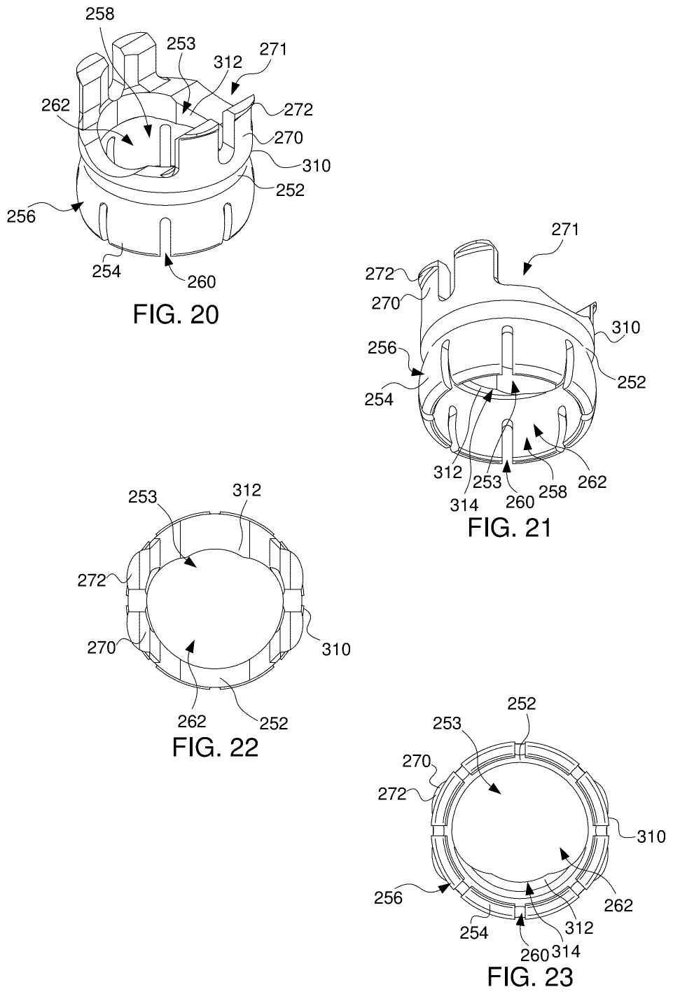

As another example, and referring now to FIGS. 20-23, an inline uniaxial clamp 310 can be the same as the transverse uniaxial clamp 250 in many respects. For example, the inline uniaxial clamp 310 can include the main body 252, the fingers 254, the convex outer engaging surfaces 256, the concave inner clamping surfaces 258, the gaps 260, and the socket 262. However, the inline uniaxial clamp motion limiting protrusion 312 with its engaging surface 314 can be positioned on a side of the clamp that is between the two pairs of flanges 270 (and thus between the two tabs 192 of the assembly head 180), rather than adjacent to a pair of the flanges 270. This can produce a movement pattern of the shank 142 relative to the inline uniaxial clamp 310 and the assembly head 180 that is an inline uniaxial movement pattern, rather than a transverse uniaxial movement pattern. Such a movement pattern can be produced by the engaging surface 314 of the inline uniaxial clamp motion limiting protrusion 312 contacting the engaging surface 164 of the shank head 160 if the pivoting movement attempts to go beyond movement allowed for the transverse uniaxial movement pattern. As with the transverse uniaxial movement pattern from the clamp 250, the inline uniaxial movement pattern from the clamp 310 may only allow movement in one direction from an aligned configuration (the direction opposite to the position of the motion limiting protrusion, as illustrated in FIGS. 33 and 37 discussed below. However, pivotal movement in an opposite direction can be achieved by rotating the position of the uniaxial clamp by one hundred and eighty degrees around a main axis of the clamp and the assembly head 180 (by either rotating the clamp relative to the assembly head or rotating the clamp and assembly head together).

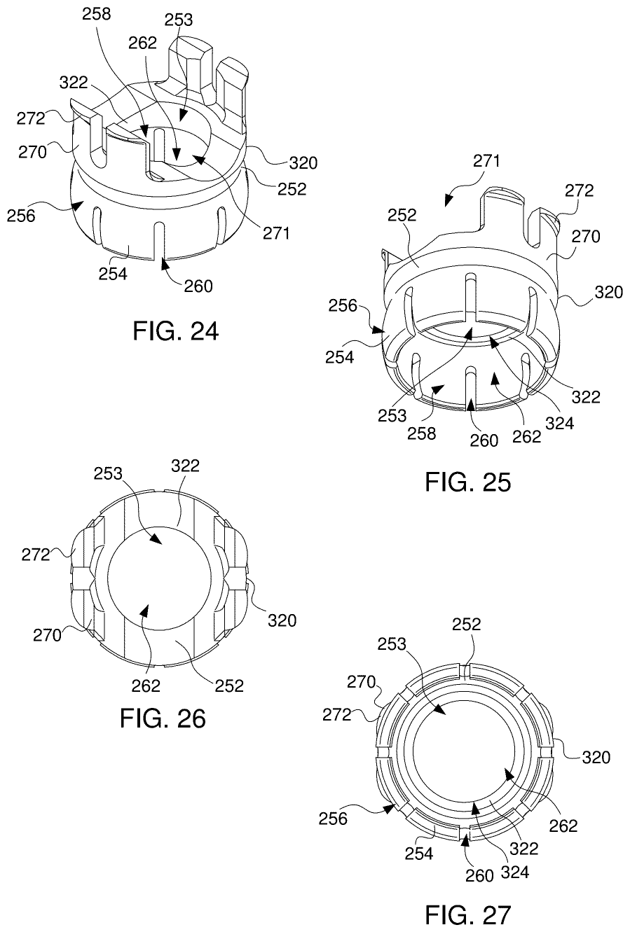

As yet another example, and referring now to FIGS. 24-27, a monoaxial clamp 320 can be the same as the transverse uniaxial clamp 250 in many respects. For example, the monoaxial clamp 320 can include the main body 252, the fingers 254, the convex outer engaging surfaces 256, the concave inner clamping surfaces 258, the gaps 260, and the socket 262. However, the monoaxial clamp motion limiting protrusion 322 with its engaging surface 324 can be a full ring extending inward from the main body 252, rather than the partial ring of the transverse uniaxial clamp 250. This can produce a movement pattern of the shank 142 relative to the monoaxial clamp 320 and the assembly head 180 that is a monoaxial movement pattern, rather than a transverse uniaxial movement pattern.

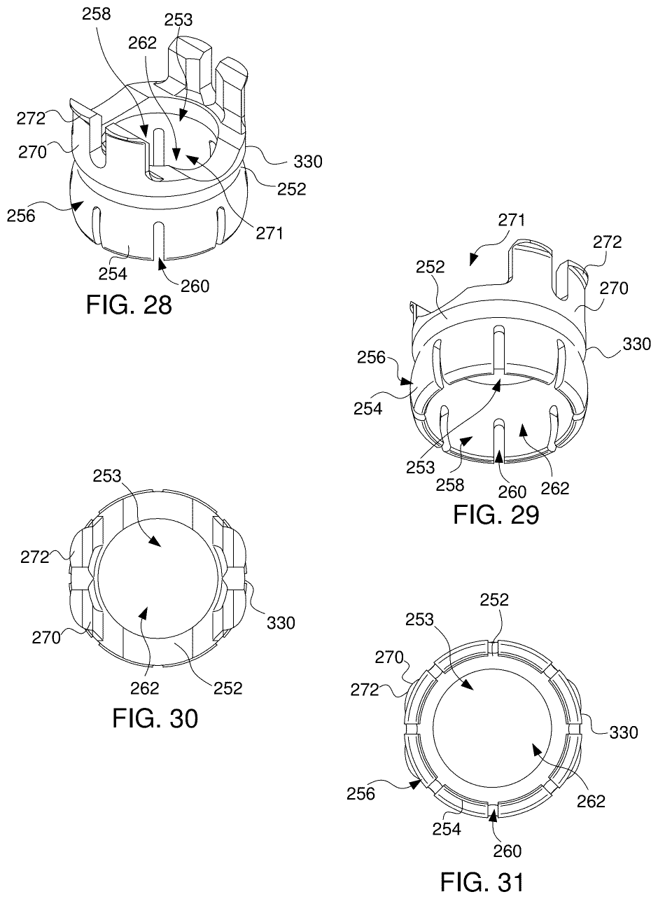

As yet another example, and referring now to FIGS. 28-31, a polyaxial clamp 330 can be the same as the transverse uniaxial clamp 250 in many respects. For example, the polyaxial clamp 330 can include the main body 252, the fingers 254, the convex outer engaging surfaces 256, the concave inner clamping surfaces 258, the gaps 260, and the socket 262. However, the polyaxial clamp 330 can omit a motion limiting protrusion and corresponding engaging surface. This can produce a movement pattern of the shank 142 relative to the inline uniaxial clamp 310 and the assembly head 180 that is a polyaxial movement pattern, rather than a transverse uniaxial movement pattern.

The motion limiting protrusions could be different shapes than those illustrated in the figures. For example, such different shapes could be other shapes that are all or a portion of a circular shape. For example, the motion limiting protrusions for uniaxial movement and the corresponding engaging surfaces could be shaped as segments of circles, as crescent shapes, or as other shapes that engage the shank head 160 to produce the uniaxial movement pattern. Similarly, the motion limiting protrusion and corresponding engaging surface for monoaxial movement could be a full circular disc or some other shape that engages the shank head 160 to produce the uniaxial movement pattern. Also, each motion limiting protrusion and each corresponding engaging surface (as well as the engaging surface of the shank head) may be split into multiple separate shapes with gaps between such separate shapes. With such different shapes, pivotal movement that is not part of the allowed movement pattern being produced can result in the engaging surface 164 of the shank head 160 contacting the shank engaging surface of the motion limiting protrusion of the clamp to inhibit such disallowed pivotal movement. However, pivotal movement within the produced movement pattern can occur without the movement being inhibited by contact between the engaging surface 164 of the shank head 160 and the shank engaging surface of the motion limiting protrusion of the clamp.

The components of the bone securing system 100 discussed herein can be formed of materials that are sufficiently strong, hard, and durable, and that are suitable for use in a living body. For example, the components may be formed of titanium alloys. However, other alternative materials may be used, such as polymer materials, composite materials, and/or other metals. Also, dimensions of the components may be altered to yield desirable properties, such as desirable strength and flexibility properties. For example, thicknesses in different areas of the components may be altered to provide desired balance of strength and flexibility for different features of the components. Manufacturing techniques for making the components of the bone securing system 100 may include standard techniques for making and post-processing parts for surgical implants, such as molding techniques, additive techniques (such as 3D printing), and/or subtractive techniques (such as milling, drilling, grinding, polishing, etc.). Also, each of the assembly components, including the shank 142, the assembly head 180, the clamps including the clamp 250 and the other clamps, and the set screw 220 can each be a monolithic part. However, in alternative implementations, one or more of these components may be made of multiple parts that are permanently or temporarily secured together.

### III. Use of a Bone Securing System

Use of the bone securing system 100 will now be discussed. Referring to FIGS. 6-7, an example of use of a bone securing system will be discussed primarily with reference to a bone securing system 100 that includes the transverse uniaxial clamp 250. However, as discussed herein, the bone securing system 100 that another clamp, such as the inline uniaxial clamp 310, the monoaxial clamp 320, or the polyaxial clamp 330 may be used in a similar manner, although such other clamps will allow different types of pivotal movement patterns, as discussed above.

FIGS. 6-7 illustrate a separated open configuration, wherein components of the head assembly 144 are separated from the shank 142. In some uses, the shank may be secured to a bone 150 while it is separated from the shank 142. In other uses, the shank 142 may be secured to a bone 150 after it is joined with components of the head assembly 144. As illustrated, the head assembly 144 can initially include the assembly head 180 and the clamp 250.

In the separated open configuration, the clamp 250 can be positioned in the assembly head 180 in an open position, wherein the protrusions 272 of the flanges 270 of the clamp 250 can extend into the proximal groove 196 of the assembly head 180 to temporarily hold the clamp 250 in place relative to the assembly head 180. The clamp 250 can be placed in such a position by inserting the clamp 250 in a distal direction through the central hole 182 of the assembly head 180. Thus, the clamp 250 can pass between the tabs 192 and at least partially through the body 184 and at least partially into the distal cavity 186 of the assembly head 180. The flanges 270 can bend inwardly toward each other to allow them to pass through the assembly head 180 to the open position. Likewise, the fingers 254 of the clamp 250 can bend inwardly toward each other to allow them to pass through the assembly head 180 to the open position.

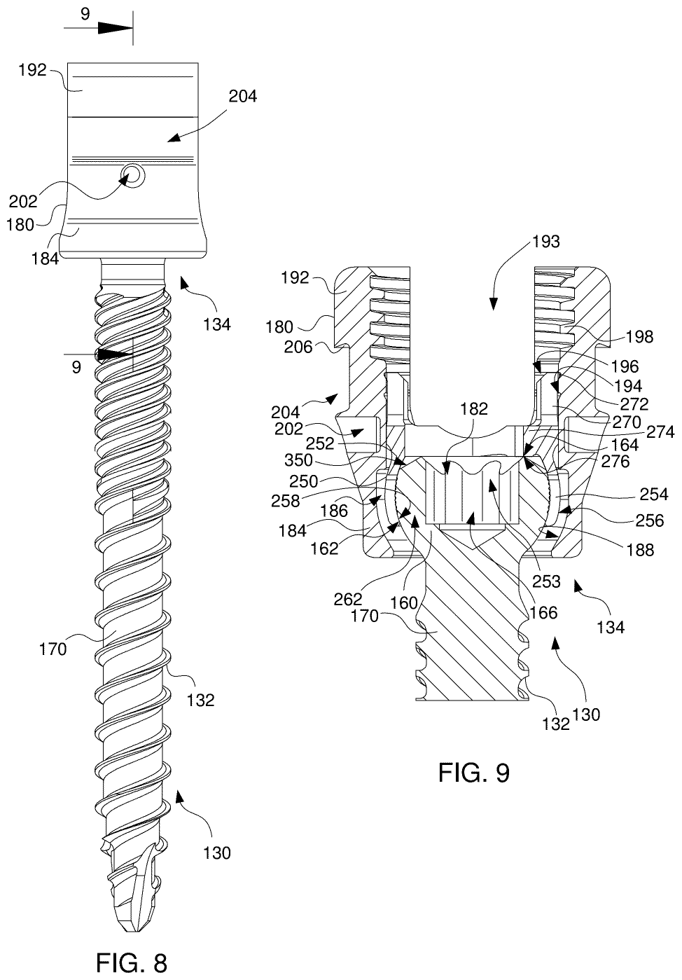

With the clamp 250 and the assembly head 180 in the open position relative to each other, the head assembly 134 can be positioned onto the shank head 160, with the shank head 160 being at least partially received into the socket 262 of the clamp 250. With the clamp 250 in the open position relative to the assembly head 180, the fingers 254 can be positioned in the distal cavity 186 of the assembly head 180 where the fingers have room to expand outward relative to each other. Thus, as the shank head 160 begins to enter the socket 262, with the shank head 160 moving proximally relative to the clamp 250, the shank head 160 can press the fingers 254 of the clamp 250 to bend outward relative to each other to expand the distal end of the socket 262 of the clamp 250, allowing the shank head 160 to enter the socket 262. This can result in the mounted open configuration illustrated in FIGS. 8-9. Thus, the head assembly 144 can be designed to be mounted on the shank head 160 without the entire extension being passed through the assembly head.

In the mounted open configuration of FIGS. 8-9, the clamp 250 may not limit pivotal movement of the shank 132 to the defined movement pattern for the clamp 250. This is because the shank head 160 may not be securely held in the socket 262 because the fingers 254 of the clamp 250 are allowed to bend outwardly to allow movement of the shank head 160 out of the socket. However, as discussed below, the clamp 250 can limit the pivotal movement of the shank 142 in the closed position of the mounted closed configuration of FIG. 11.

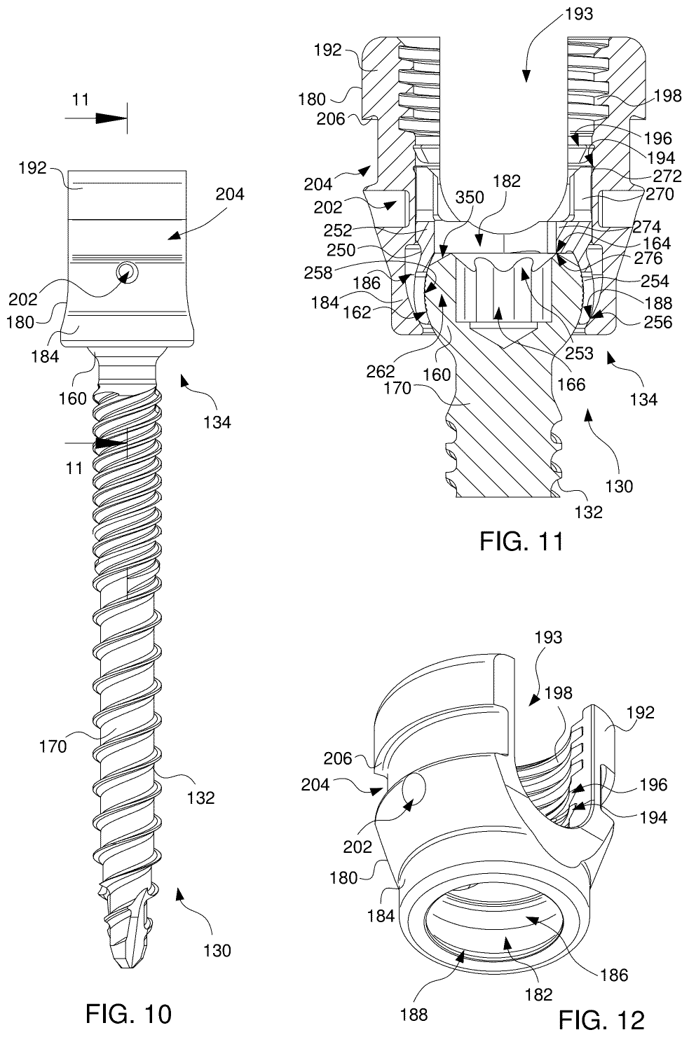

With the first bone anchor assembly 130 in the mounted open configuration of FIGS. 8-9, an instrument can be used to pull the assembly head 180 proximally and to push the clamp 250 distally. For example, such an instrument can engage the opposing holes 202 and/or the opposing recesses 204 in the assembly head 180 to pull the assembly head 180, and the instrument can engage the tops of the flanges 270 and/or proximally facing surfaces of the clamp 250 between the flanges 270 to push the clamp 250. For example, the instrument may work similarly to a rod reduction tool. The proximal pulling of the assembly head 180 and the distal pushing of the clamp 250 can result in the assembly head 180 being moved proximally relative to the clamp 250 so that the clamp 250 and the shank head 160 slide distally in the assembly head 180 until the protrusions 272 of the flanges 270 of the clamp 250 are forced out of the proximal grooves 196 in the assembly head and spring into the distal grooves 194 of the assembly head. This movement results in the bone anchor assembly being in the mounted closed configuration illustrated in FIGS. 10-11.

In the mounted closed configuration, the head assembly 134 can retain the shank head 160 in the socket 262 of the clamp 250. Specifically, in that configuration, the convex outer engaging surface 256 of each finger 254 can be engaged by the clamp engaging surface 188 of the assembly head 180 to inhibit outward bending movement of the fingers 254. Additionally, the concave inner clamping surface 258 of each finger 254 of the clamp 250 can extend around and engage the clamped surface 162 of the shank head 160 to inhibit movement of the shank head 160 out of the socket 262. Thus, in the mounted closed configuration, the clamp 250 can hold the shank head 160 in the assembly head 180.

Additionally, in the mounted closed configuration, the clamp 250 can limit pivotal movement of the shank 132 relative to the clamp 250 and relative to the assembly head 180 to the movement pattern defined by the clamp 250.

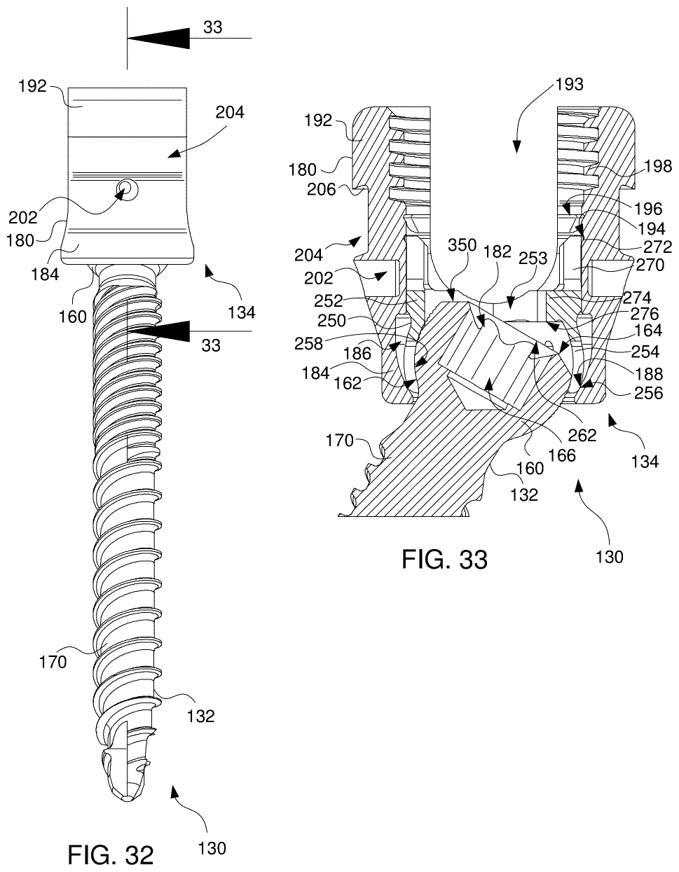

Accordingly, in the illustrated example, a transverse unilateral movement pattern can be allowed. This can include pivoting the shank 132 and the head assembly 144 relative to each other in a plane that is perpendicular to the direction of the channel 193. For example, this can include pivoting the shank 132 and the head assembly 144 relative to each other from the aligned position illustrated in FIGS. 10-11 to the angled position illustrated in FIGS. 32-33 (which may be at a thirty degree angle). As can be seen in at least FIGS. 11 and 33, the shank engaging surface 276 of the clamp 250 can define an engagement plane (the plane that is coplanar with the engaging surface 276 in the example of FIGS. 11 and 33). A first portion of the engaging surface 164 of the shank head 160 (the portion on the left in FIG. 33) can pass through that plane to allow the pivoting illustrated in FIG. 33. However, a second portion of the engaging surface 164 of the shank head 160 (the portion on the right in FIG. 33) can be blocked from passing through that plane by the engagement between the engaging surface 164 of the shank head 160 and the shank engaging surface 276 of the clamp 250. For a polyaxial clamp (such as for the polyaxial clamp 330), such an engagement plane may not be defined because of the lack of a similar shank engaging surface on the clamp. And for a monoaxial clamp (such as the monoaxial clamp 320), the entire engaging surface 164 of the shank head 160 may be blocked from passing through the engagement plane. In some examples, as in the example of FIGS. 32-33, the engagement plane may be perpendicular to an axial direction of the assembly head 180. However, in other examples, the engagement plane could be in some other orientation. Also, in some examples, the engagement surfaces may not be planar surfaces, but may still block pivoting in one or more directions, but allow pivoting in one or more other directions. The engagement between the engaging surface 164 of the shank head 160 and the shank engaging surface 276 of the clamp 250 can be a direct abutting engagement. Alternatively, one or more structural features may be positioned between the engaging surface 164 of the shank head 160 and the shank engaging surface 276 of the clamp 250, so that the engagement would be an indirect engagement through the one or more structural features.

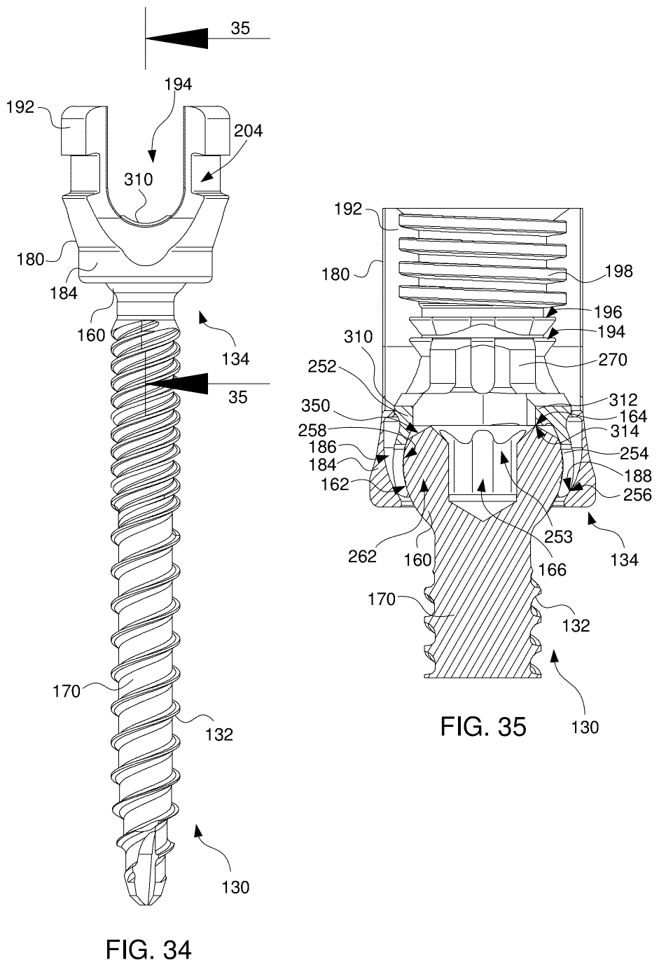

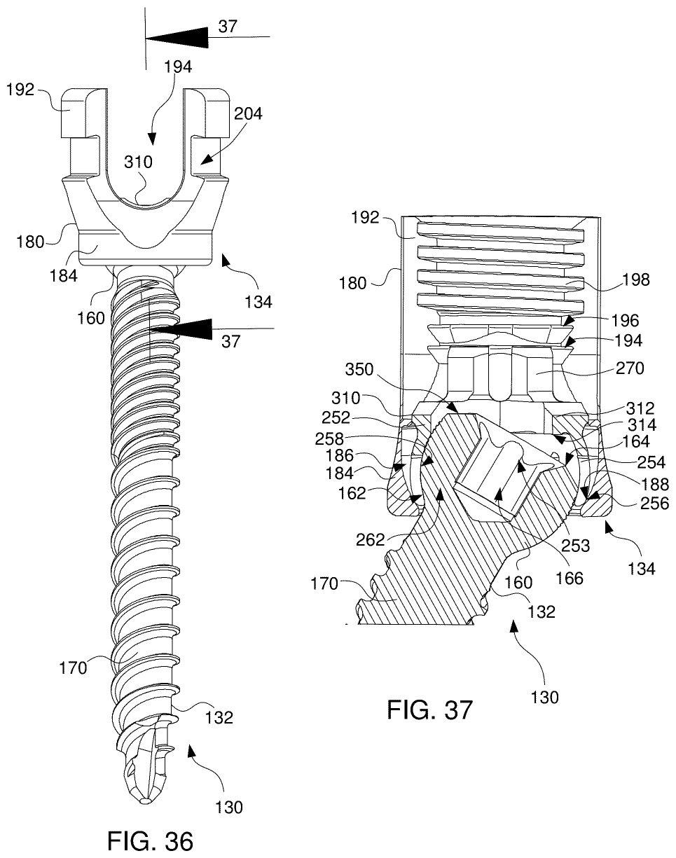

Likewise, with the inline uniaxial clamp 310 of FIGS. 20-23 in the mounted closed configuration, an inline unilateral movement pattern can be allowed. This can include pivoting the shank 132 and the head assembly 144 relative to each other in a plane that is parallel to the direction of the channel 193. For example, this can include pivoting the shank 132 and the head assembly 144 relative to each other from the aligned position illustrated in FIGS. 34-35 to the angled position illustrated in FIGS. 36-37, which can be at a pivoted angle of thirty degrees. As illustrated in the figures herein, in an implementation, the shank head 160 may define a sloped surface 350, which is neither perpendicular to nor parallel with the rotational axis of the shank 132, at least partially defines the engaging surface 164, such as where the engaging surface 164 may be an inner edge of the sloped surface 350. The sloping of the sloped surface 350 can help to keep the shank head 160 from interfering with the rod 110 when the rod is positioned in the channel 193 between the tabs 192 and the flanges 270. In addition to or instead of such a sloped surface 350, shank head may include an engaging surface perpendicular to the rotational axis of the shank 142, and/or the clamp 250 and the assembly head 180 can be designed with increased proximal-distal height between the shank head 160 in the socket 262 of the clamp 250 and the rod 110 in the channel 193 to allow sufficient distance between the shank head and the rod 110 when the shank head 160 is rotated. This can be done by increasing the height of the bodies of the clamp 250 and the assembly head 180. Such increased distance may allow greater pivoting angles of the head assembly 144 relative to the shank 132 without the shank head 160 interfering with the rod 110.

Referring to FIGS. 38-39, as an alternative, an inline uniaxial clamp 360 may include a motion limiting protrusion 362 that defines a sloped clamp engaging surface 364 that matches the sloped surface 350 of the shank head 160, so that there can be a larger surface engagement between the clamp engaging surface 354 of the clamp 360 and the engaging surface 164 of the shank head 160 (i.e., the engaging surface 164 can be a larger portion of the sloped surface 350 instead of an inner edge of the sloped surface 350). Thus, the sloped surface 350 can be a frustoconical surface shape, and the clamp engaging surface 364 may be a portion of a matching frustoconical surface shape. Also, as an alternative, the clamp engaging surface 354 can be an even larger matching surface than the one illustrated in FIG. 39, where the clamp engaging surface can extend farther outward along the sloped surface 350 of the shank head 160, possibly even extending to the outer edge of the sloped surface 350, so that an even larger area of the sloped surface 350 can form the engaging surface 164. Besides the differences in the motion limiting protrusion 362 of the clamp 360, the structure and operation of the features of the example illustrated in FIGS. 38-39 can be the same as in the example of FIGS. 34-37. The shank engaging surfaces of one or more of the other uniaxial and/or monoaxial clamps discussed herein can also be sloped to match the shank head's sloped clamp engaging surface 364 in similar ways (for example, the sloped surface of the other uniaxial clamp may be a similar portion of a frustoconical surface shape, and the sloped surface of a monoaxial clamp may be a full frustoconical surface shape (or possibly a portion thereof that can still inhibit pivoting of the shank relative to the clamp)).

With the transverse uniaxial clamp 250 or the inline uniaxial clamp 310, the clamp can be rotated one-hundred and eighty degrees around a proximal-distal axis to reverse an allowed direction of pivoting for the movement pattern to which the clamp limits pivotal movement between the shank 132 and the head assembly 144. For example, if the transverse uniaxial clamp 250 allows thirty-degree pivotal movement in a first direction from an aligned position, rotating the clamp one-hundred and eighty degrees can produce an allowed movement patter with thirty-degree pivotal movement in a second direction that is opposite to the first direction. Similarly, if the inline uniaxial clamp 310 allows thirty-degree pivotal movement in a first direction from an aligned position, rotating the clamp one-hundred and eighty degrees can produce an allowed movement pattern with thirty-degree pivotal movement in a second direction that is opposite to the first direction.

With the polyaxial clamp 330 of FIGS. 28-31 in the mounted closed configuration, a polyaxial movement pattern can be allowed. This can include pivoting the shank 132 and the head assembly 144 relative to each other around multiple axes and in multiple planes, including a plane that is parallel to the direction of the channel 193, a plane that is perpendicular to the direction of the channel 193, and other planes at different angles to the direction of the channel 193.

With the monoaxial clamp 320 of FIGS. 24-27 in the mounted closed configuration, a monoaxial movement pattern can be allowed. This can include inhibiting pivoting of the shank 132 and the head assembly 144 relative to each other.

In addition to the pivotal movements discussed above, with the transverse uniaxial clamp 250, the inline uniaxial clamp 310, the monoaxial clamp 320, or the polyaxial clamp 330, the shank 132 can be allowed to rotate around its longitudinal axis relative to the head assembly 144 in the mounted closed configuration, such as to screw the shank 132 into or out of a bone 150.

Besides the limiting of pivotal movement of the shank 132 relative to the head assembly 144 by the motion limiting protrusions of the clamps, the pivotal movement may also be limited by other engagements, such as by engagement of the extension 170 of the shank 142 with the distal end of the clamp and/or the assembly head 180 (see FIG. 33). This may include having features to engage the shank 142 and keep the pivotal movement from being so great that the shank head 160 interferes with the rod 110 when the rod is seated in the channel 193 between the tabs 192 of the assembly head 180 and the flanges 270 of the clamp.

With the shank 132 secured in bone 150, and with the first bone anchor

assembly 130 in the mounted closed configuration with the head assembly 134 being pivoted to a desired angle (which may be aligned so that the angle is zero) relative to the shank 132, the rod 110 can be positioned in the channel 193 between the tabs 192 of the assembly head 180 and the channel 271 between pairs of the flanges 270 of the clamp 250. The rod 110 can be moved distally relative to the head assembly 134 until it rests on the base of the channel 271 between pairs of the flanges 270 of the clamp 250 (resting on that base, on opposite sides of the hole passing axially in a proximal-distal direction through the main body 252 of the clamp 250). This may include performing rod reduction using a rod reduction instrument.

With the rod 110 positioned in the channel 193, the set screw 220 can be screwed into the female threads 198 of the tabs 192 of the assembly head 180, as illustrated in the locked position and configuration of FIG. 5. For example, the set screw 220 can be tightened using an instrument to engage the central tool-receiving aperture in the break-off head 228. The set screw 220 can be tightened until the break-off head 228 breaks off from the set screw 220. As the set screw 220 is tightened, the set screw 220 can push the rod 110 distally relative to the assembly head 180 and can push the rod 110 against the clamp 250 on opposite ends of the channel 271 in the clamp 250 at the base of the channel 271, thereby pressing the clamp 250 distally relative to the assembly head 180. This pressing of the clamp 250 distally relative to the assembly head 180 can press the convex outer engaging surfaces of the fingers 254 of the clamp 250 against the clamp engaging surface 188 of the assembly head 180. This pressing between the clamp 250 and the assembly head 180 can press the fingers 254 inwardly towards each other, thereby pressing the concave inner clamping surfaces 258 of the fingers 254 of the clamp 250 against the clamped surface 162 of the shank head 160. This increased clamping force on the shank head 160 can lock the shank head 160 into place relative to the head assembly 144, which may include inhibiting any pivotal movement of the shank 142 relative to the head assembly 144. This inhibiting of the movement can be enhanced by surface textures on the clamped surface 162 of the shank head 160 and/or the concave inner clamping surface 258 of the clamp 250. For example, such textures may include grooves, ridges, or other small protrusions and/or apertures in the surfaces.

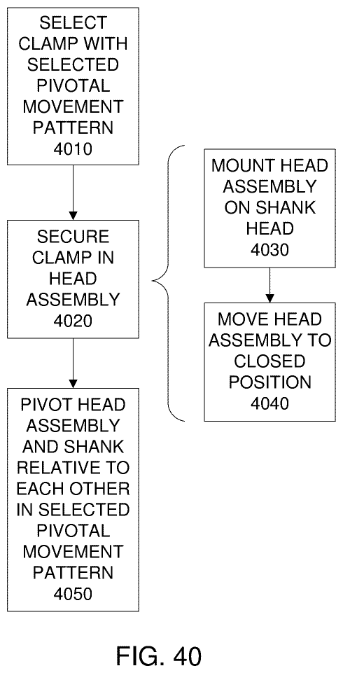

Thus, referring to FIG. 40, the techniques discussed herein can include one or more apparatus assembly techniques in which a head clamp may be selected 4010 from multiple different available head clamps. The selection of a clamp may include just selecting the clamp itself, or selecting an assembly that includes the selected clamp, from among multiple different head assemblies. The different available head clamps can be designed to produce different pivotal movement patterns between head assemblies in which the head clamps are secured and shanks that are secured to the head assemblies using the head clamps. The selected head clamp can be designed to produce a selected pivotal movement pattern. The multiple different head assemblies can include different clamps having different designs, but with the same assembly head design.

The techniques may include securing 4020 a head assembly on a shank head of a shank in a motion limiting configuration. The shank can be designed to be secured in bone. The head assembly can include the selected head clamp positioned at least partially in an assembly head in the motion limiting configuration. The head assembly can be configured to secure the shank to a rod. The securing 4020 of the head assembly can include receiving at least a portion of the shank head in the selected head clamp. The selected head clamp can include an engaging surface that is shaped and positioned to engage the engaging surface of the shank head to limit pivotal movement of the shank relative to the selected head clamp to the selected pivotal movement pattern while in the motion limiting configuration.

The securing 4020 may include mounting 4020 the head assembly on the shank head with the head assembly in an open position wherein the shank head is moveable into and out of the selected head clamp, and moving 4040 the head assembly to a closed position in which the head assembly inhibits movement of the shank head out of the selected head clamp. The motion limiting configuration can include the head assembly being in the closed position.

The technique can further include pivoting 4050 the head assembly and the shank relative to each other within the selected pivotal movement pattern while the selected head clamp and the shank are in the motion limiting configuration. The pivoting 4050 can include pivoting at least a portion of the engaging surface of the shank head away from at least a portion of the engaging surface of the selected head clamp while the at least a portion of the engaging surface of the shank head and the at least a portion of the engaging surface of the head clamp face toward each other.

The technique can further include locking the shank relative to the head assembly in a locked configuration that inhibits pivoting of the shank and the head assembly relative to each other. The locking can include positioning a rod in the head assembly, and securing the rod in the head assembly with a set screw, which can apply a locking force to lock the shank in a pivotal position relative to the head assembly. As discussed herein, the set screw can then be part of the head assembly. Also, the shank, the assembly head, the clamp, and the set screw can all be part of a bone anchor assembly. One or more bone anchor assemblies may be secured to one or more rods to form a bone securing system. If there are multiple bone anchor assemblies, all or part of the assembly technique discussed above may be repeated for each bone anchor assembly in the bone securing system.

This assembly technique can also be part of a surgical procedure, which can include securing the shank to a bone either before or after the head assembly is mounted on the shank head. The pivoting 4050 can be performed while the shank is secured to the bone.

### IV. Aspects of the Bone Securing System

According to one aspect, a bone securing system can include a shank that is designed to be secured to bone. The shank can include a shank head and an extension extending from the shank head. Also, the shank head can include an engaging surface. The bone securing system can also include a head assembly, which can include an assembly head and a head clamp. The assembly head can define a central hole therethrough extending in a proximal direction and in a distal direction that is opposite the proximal direction. The assembly head can define a channel transverse to the central hole. Also, the assembly head can include tabs extending in the proximal direction on opposite sides of the channel and the central hole. The head clamp can be designed to be positioned at least partially within the central hole. The head clamp can be designed to receive the shank head and to clamp onto the shank head to secure the shank head in position at least partially in the assembly head, with the extension extending away from the head assembly. The head clamp can include an engaging surface that is shaped and positioned to engage the engaging surface of the shank head to limit pivotal movement of the shank relative to the head clamp to a movement pattern. At least a portion of the engaging surface of the head clamp can face in the distal direction toward the shank head.

The engaging surface of the shank head can face away from the extension. Also, the head clamp can be designed to be fixed to the assembly head while the head clamp is positioned at least partially within the central hole, and the engaging surface of the head clamp can be shaped and positioned to engage the engaging surface of the shank head to limit pivotal movement of the shank relative to the assembly head.

The head assembly can be designed to allow pivotal movement of the shank in the movement pattern relative to the head clamp. As an example, the movement pattern can be a uniaxial movement pattern.

The head assembly can be designed to be mounted on the shank head without all of the extension being passed into the assembly head.

The head clamp can be a collar with a central hole passing therethrough, and the engaging surface of the head clamp can form a shape that is at least a portion of a circular shape, such as at least a portion of a ring shape. The engaging surface may be on a shoulder that faces in the distal direction when the head clamp is positioned at least partially in the assembly head.

The assembly head can be designed to move proximally into a closed position relative to the head clamp to secure the shank head relative to the head clamp and the assembly head, while still allowing the movement pattern of the shank relative to the head clamp and the assembly head. The channel can be designed to receive a rod, and the head assembly can be designed to secure the rod to the shank. The securing of the rod to the shank can apply pressure to lock the shank relative to the head clamp and the assembly head.

The head clamp can be a first head clamp, the engaging surface of the first head clamp can be a first engaging surface, and the movement pattern can be a first movement pattern. The bone securing system can further include a second head clamp designed to be positioned at least partially within the central hole instead of the first head clamp. The second head clamp can be designed to receive the shank head and to clamp onto the shank head to secure the shank head in position at least partially in the assembly head with the extension extending away from the head assembly. The second head clamp can be shaped differently from the first head clamp to limit movement of the shank to a second movement pattern that is different from the first movement pattern. As an example, the first movement pattern can be a uniaxial movement pattern, and the second movement pattern can be either a different uniaxial movement pattern from the first movement pattern, a polyaxial movement pattern, or a monoaxial movement pattern.

In yet another aspect, a shank can be designed to be secured to bone. The shank can include a shank head and an extension extending from the shank head. The shank head can include an engaging surface that faces away from the extension. The bone securing system can also include a head assembly designed to secure the shank head to a rod. The head assembly can include an engaging surface that is shaped and positioned to engage the engaging surface of the shank head to limit pivotal movement of the shank relative to the engaging surface of the head assembly, but to allow some pivotal movement of the shank relative to the engaging surface of the head assembly. The engaging surface of the head assembly can face toward the engaging surface of the shank head.

The engaging surface of the shank head can be on an end of the shank head opposite the extension. The engaging surface of the head assembly can form a shape that is at least a portion of a circular shape, such as at least a portion of a ring shape.

In yet another aspect, a bone securing system can include a shank designed be secured to bone. The shank can include a shank head and an extension extending from the shank head. The shank head can include an engaging surface that faces away from the extension. The bone securing system can also include multiple different head assemblies that each allow a different pivotal movement pattern between the head assembly and the shank. A first head assembly of the head assemblies can include a first engaging surface that is shaped and positioned to engage the engaging surface of the shank head to limit pivotal movement of the shank relative to the first engaging surface of the first head assembly to a first movement pattern.

As an example, the first movement pattern can be a uniaxial movement pattern.

A second head assembly of the head assemblies can include a second engaging surface that is shaped and positioned to engage the engaging surface of the shank head to limit pivotal movement of the shank relative to the second engaging surface of the second head assembly to a second movement pattern that is either a monoaxial movement pattern or a polyaxial movement pattern.

The first head assembly may include an assembly head defining a central hole therethrough and a channel transverse to the central hole. The assembly head can include proximally extending tabs on opposite sides of the channel. The head assembly can also include a head clamp designed to be positioned at least partially within the central hole. The head clamp can be designed to receive the shank head and to clamp onto the shank head to secure the shank head in position at least partially in the assembly head with the extension extending away from the head assembly. The head clamp can include the first engaging surface.

The subject matter defined in the appended claims is not necessarily limited to the benefits described herein. A particular implementation of the invention may provide all, some, or none of the benefits described herein. Although acts for the various techniques are described herein in a particular, sequential order for the sake of presentation, it should be understood that this manner of description encompasses rearrangements in the order of operations, unless a particular ordering is required. For example, operations described sequentially may in some cases be rearranged or performed concurrently. Also, features can be omitted, and different features can be combined any way that is not disallowed by the features themselves or the language describing them in this description.

While particular examples are discussed above, it will be understood by those skilled in the art that various changes in form and details may be made therein without departing from the spirit and scope of the invention.

I/we claim:

1. A bone securing system comprising:
a shank that is designed to be secured to bone, the shank comprising a shank head and an extension extending from the shank head, the shank head comprising an engaging surface; and
a head assembly comprising:
an assembly head defining a central hole therethrough extending in a proximal direction and in a distal direction that is opposite the proximal direction, the assembly head defining a channel transverse to the central hole, the assembly head comprising tabs extending in the proximal direction on opposite sides of the channel and the central hole; and
a head clamp designed to be positioned at least partially within the central hole, the head clamp being designed to receive the shank head and to clamp onto the shank head to secure the shank head in position at least partially in the assembly head with the extension extending away from the head assembly, the head clamp comprising an engaging surface that is shaped and positioned to engage the engaging surface of the shank head to limit pivotal movement of the shank relative to the head clamp to a movement pattern, at least a portion of the engaging surface of the head clamp facing in the distal direction toward the shank head.

2. The bone securing system of claim 1, wherein the engaging surface of the shank head faces away from the extension.

3. The bone securing system of claim 1, wherein the head clamp is designed to be fixed to the assembly head while the head clamp is positioned at least partially within the central hole, and the engaging surface of the head clamp is shaped and positioned to engage the engaging surface of the shank head to limit pivotal movement of the shank relative to the assembly head.

4. The bone securing system of claim 1, wherein the head assembly is designed to allow pivotal movement of the shank in the movement pattern relative to the head clamp.

5. The bone securing system of claim 1, wherein the movement pattern is a uniaxial movement pattern.

6. The bone securing system of claim 1, wherein the head assembly is designed to be mounted on the shank head without all of the extension being passed into the assembly head.

7. The bone securing system of claim 1, wherein the head clamp is a collar with a central hole passing therethrough.

8. The bone securing system of claim 7, wherein the engaging surface of the head clamp forms a shape that is at least a portion of a circular shape.

9. The bone securing system of claim 7, wherein the engaging surface is on a shoulder that faces in the distal direction when the head clamp is positioned at least partially in the assembly head.

10. The bone securing system of claim 1, wherein the assembly head is designed to move proximally into a closed position relative to the head clamp to secure the shank head relative to the head clamp and the assembly head, while still allowing the movement pattern of the shank relative to the head clamp and the assembly head.

11. The bone securing system of claim 1, wherein the channel is designed to receive a rod, and the head assembly is designed to secure the rod to the shank, the securing of the rod to the shank applying pressure to lock the shank relative to the head clamp and the assembly head.

12. The bone securing system of claim 1, wherein the head clamp is a first head clamp, the engaging surface of the first head clamp is a first engaging surface, the movement pattern is a first movement pattern, and the bone securing system further comprises:
a second head clamp designed to be positioned at least partially within the central hole instead of the first head clamp, the second head clamp being designed to receive the shank head and to clamp onto the shank head to secure the shank head in position at least partially in the assembly head with the extension extending away from the head assembly, the second head clamp being shaped differently from the first head clamp to limit movement of the shank to a second movement pattern that is different from the first movement pattern.

13. The bone securing system of claim 12, wherein the first movement pattern is a uniaxial movement pattern, and wherein the second movement pattern is either a different uniaxial movement pattern from the first movement pattern, a polyaxial movement pattern, or a monoaxial movement pattern.

14. A bone securing system comprising:
a shank designed to be secured to bone, the shank comprising a shank head and an extension extending from the shank head, the shank head comprising an engaging surface that faces away from the extension; and
a head assembly designed to secure the shank head to a rod, the head assembly comprising an engaging surface that is shaped and positioned to engage the engaging surface of the shank head to limit pivotal movement of the shank relative to the engaging surface of the head assembly, but to allow some pivotal movement of the shank relative to the engaging surface of the head assembly, the engaging surface of the head assembly facing toward the engaging surface of the shank head.

15. The bone securing system of claim 14, wherein the engaging surface of the shank head is on an end of the shank head opposite the extension.

16. The bone securing system of claim 14, wherein the engaging surface of the head assembly forms a shape that is at least a portion of a circular shape.

17. A method comprising:
selecting a selected head clamp from multiple different available head clamps, the different available head clamps being designed to produce different pivotal movement patterns between head assemblies in which the head clamps are secured and shanks that are secured to the head assemblies using the head clamps, the selected head clamp being designed to produce a selected pivotal movement pattern;
securing a head assembly on a shank head of a shank in a motion limiting configuration, the shank being designed to be secured in bone, the head assembly comprising the selected head clamp positioned at least partially in an assembly head in the motion limiting configuration, the head assembly being configured to secure the shank to a rod, the securing of the head assembly comprising receiving at least a portion of the shank head in the selected head clamp, and the selected head clamp comprising an engaging surface that is shaped and positioned to engage the engaging surface of the shank head to limit pivotal movement of the shank relative to the selected head clamp to the selected pivotal movement pattern while in the motion limiting configuration; and
pivoting the head assembly and the shank relative to each other within the selected pivotal movement pattern while the selected head clamp and the shank are in the motion limiting configuration, the pivoting comprising pivoting at least a portion of the engaging surface of the shank head away from at least a portion of the engaging surface of the selected head clamp while the at least a portion of the engaging surface of the shank head and the at least a portion of the engaging surface of the head clamp face toward each other.

18. The method of claim 17, further comprising locking the shank relative to the head assembly in a locked configuration that inhibits pivoting of the shank and the head assembly relative to each other.

19. The method of claim 17, wherein the securing comprises mounting the head assembly on the shank head with the head assembly in an open position wherein the shank head is moveable into and out of the selected head clamp, and moving the head assembly to a closed position in which the head assembly inhibits movement of the shank head out of the selected head clamp, wherein the motion limiting configuration comprises the head assembly being in the closed position.

20. The method of claim 17, further comprising securing the shank to a bone, wherein the pivoting is performed while the shank is secured to the bone.
